(19) United States
(12) Patent Application Publication
(10) Pub. No.: US 2026/0248740 A1
(43) Pub. Date: Aug. 27, 2026

(54) Sustainable Biopolymer Compositions for Skin-Contact Wearable Articles

(71) Applicant: Zhirong Yu, Los Angeles, CA (US)

(72) Inventor: Zhirong Yu, Los Angeles, CA (US)

(21) Appl. No.: 19/545,647

(22) Filed: Feb. 20, 2026

Related U.S. Application Data
(60) Provisional application No. 63/761,586, filed on Feb. 21, 2025.

(51) Int. Cl. A61K 9/70; A61K 8/02; A61K 8/34; A61K 8/49; A61K 8/73; A61Q 19/00

(52) U.S. Cl. CPC A61K 9/7053; A61K 8/0208; A61K 8/345; A61K 8/4913; A61K 8/732; A61Q 19/007

(57) ABSTRACT

A sustainable biopolymer composition for skin-contact applications comprises a polymer matrix comprising a gelling agent selected from the group consisting of gelatin, agar, and combinations thereof. The composition comprises a humectant system comprising sucrose. The composition comprises water. The polymer matrix, the humectant system, and the water form a homogeneous gel network. The composition is configured to be shaped into a skin-contact article and dehydrated to form a biodegradable adhesive material. Multi-layer sustainable biopolymer articles comprising structural backing layers and skin-contact adhesive layers with varying glycerin content are also provided. Methods of manufacturing the sustainable biopolymer material include dissolving the gelling agent in water, adding sucrose, heating the solution, pouring the solution into a three-dimensional mold, and dehydrating the molded solution by air drying to produce a first surface having greater tackiness and a second surface having greater structural stability.

### CROSS-REFERENCE TO RELATED APPLICATIONS

This application claims priority to U.S. Provisional Application No. 63/761,586, filed Feb. 21, 2025, which is hereby incorporated by reference in its entirety.

### FIELD OF INVENTION

The present disclosure relates to sustainable biopolymer compositions for skin-contact applications, and more particularly to biodegradable adhesive materials comprising polysaccharide or protein-based polymer networks with humectant systems for use in activewear elements, undergarments, and therapeutic skin patches.

### BACKGROUND

Skin-contact wearable articles encompass a broad category of products designed for direct application to the human body, including undergarments, activewear elements, therapeutic patches, bandages, and cosmetic applications. These products serve various functions such as providing coverage, delivering active ingredients to the skin, protecting against friction or chafing, and supporting wound healing processes.

Conventional skin-contact wearable articles have traditionally relied on synthetic materials and petroleum-based adhesives for their construction. Adhesive materials used in products such as nipple covers, skin patches, bandages, anti-chafing products, and similar applications often comprise synthetic polymers derived from petrochemical sources. While these synthetic materials can provide adequate adhesion and structural properties, they may present certain drawbacks in terms of skin compatibility and environmental impact.

Some users experience skin irritation or sensitivity reactions when using products containing synthetic adhesives or polymer materials. The skin-material interface presents challenges in balancing adhesion strength with gentle removal and comfort during extended wear periods. Products that adhere too strongly may cause discomfort or skin damage upon removal, while products with insufficient adhesion may fail to remain in place during physical activity or normal daily movement.

Environmental considerations have become increasingly relevant in the development of consumer products, including skin-contact wearable articles. Many existing skin-contact adhesive products are designed for single use and contribute to waste accumulation after disposal. Synthetic polymer materials may persist in the environment for extended periods due to their resistance to biodegradation. The accumulation of non-biodegradable waste from disposable consumer products has prompted interest in developing alternative materials derived from renewable sources.

Biopolymer-based materials derived from natural sources offer potential alternatives to synthetic materials for various applications. Polysaccharides, such as agar and starch, and proteins, such as gelatin, can form gel networks with varying degrees of adhesion, flexibility, and moisture management properties. These naturally-derived polymers are generally biodegradable and may be obtained from renewable plant or animal sources.

The incorporation of humectants and plasticizers into biopolymer matrices can modulate the mechanical and adhesive characteristics of the resulting materials. Humectants such as sugars and polyols can attract and retain moisture, influencing the flexibility, tack, and skin-feel of biopolymer compositions. Plasticizers can reduce brittleness and increase the conformability of biopolymer films and gels.

However, formulating biopolymer compositions that achieve suitable adhesion to skin while maintaining structural integrity, comfort, and biodegradability presents ongoing challenges. The balance between adhesion strength, ease of removal, reusability, and environmental degradability involves complex interactions between the polymer matrix, humectant system, and processing conditions. Multi-layer structures incorporating different compositions may offer approaches to achieving combinations of properties that are difficult to obtain with single-layer materials.

Accordingly, there remains interest in developing sustainable material compositions for skin-contact wearable articles that address environmental considerations while providing desirable functional properties for applications including activewear elements, undergarments, therapeutic patches, and related products.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a sustainable biopolymer composition for skin-contact applications is provided. The composition includes a polymer matrix comprising a gelling agent selected from the group consisting of gelatin, agar, and combinations thereof. The composition includes a humectant system comprising sucrose. The composition includes water. The polymer matrix, the humectant system, and the water form a homogeneous gel network. The composition is configured to be shaped into a skin-contact article and dehydrated to form a biodegradable adhesive material.

According to other aspects of the present disclosure, the sustainable biopolymer composition may include one or more of the following features. The gelling agent may comprise agar. The gelling agent may comprise gelatin. The humectant system may further comprise at least one polyol selected from the group consisting of glycerol, sorbitol, and combinations thereof. The at least one polyol may comprise glycerin present in an amount ranging from 5 g to 10 g. The humectant system may further comprise propylene glycol present in an amount ranging from 3 g to 6 g. The composition may further comprise xanthan gum present in an amount ranging from 0.5 g to 1 g. The composition may further comprise pyrrolidone carboxylic acid or a salt thereof present in an amount ranging from 0.05 wt % to 15 wt %. The composition may further comprise a bioadhesive polymer component selected from the group consisting of chitosan derivatives, carboxyl-containing polymers, and combinations thereof. The composition may further comprise at least one functional additive selected from the group consisting of fragrance compounds, menthol, nourishing oils, skin care actives, pimple treatment actives, moisturizing ingredients, and preservatives. The at least one functional additive may comprise menthol configured to provide cooling functionality to the skin-contact article.

According to another aspect of the present disclosure, a multi-layer sustainable biopolymer article for skin-contact applications is provided. The article includes a first layer comprising a structural backing layer. The structural backing layer comprises a first composition including a gelling agent selected from the group consisting of gelatin, agar, and combinations thereof, and sucrose. The first layer is in a dehydrated state. The article includes a second layer comprising a skin-contact adhesive layer. The skin-contact adhesive layer comprises a second composition including a gelling agent selected from the group consisting of gelatin, agar, and combinations thereof, sucrose, and glycerin. The glycerin content of the second layer is greater than a glycerin content of the first layer. The second layer is configured to contact skin and provide adhesion.

According to other aspects of the present disclosure, the multi-layer sustainable biopolymer article may include one or more of the following features. The glycerin content of the second layer may range from 10 g to 20 g. The article may further comprise a starch-based durability layer. The starch-based durability layer may comprise tapioca starch present in an amount ranging from 2 g to 10 g combined with agar and sucrose. The article may further comprise a fabric layer positioned between the starch-based durability layer and the skin-contact adhesive layer. The fabric layer may comprise a transparent fabric selected from the group consisting of nylon tulle, power mesh, spandex mesh, and pantyhose material. The article may further comprise a bioplastic layer. The bioplastic layer may comprise starch present in an amount ranging from 2 g to 10 g and an acid selected from the group consisting of citric acid and fruit acid present in an amount ranging from 3 g to 5 g.

According to another aspect of the present disclosure, a method of manufacturing a sustainable biopolymer material for skin-contact applications is provided. The method includes dissolving a gelling agent selected from the group consisting of agar and gelatin in water to form a solution. The method includes adding sucrose to the solution. The method includes heating the solution. The method includes pouring the solution into a three-dimensional mold. The method includes dehydrating the molded solution by air drying to form the sustainable biopolymer material. The dehydrating produces a first surface having greater tackiness and a second surface having greater structural stability.

According to other aspects of the present disclosure, the method may include one or more of the following features. The method may further comprise cooling the solution to approximately 55 degrees Celsius and adding at least one plasticizer to the cooled solution while stirring prior to pouring the solution into the three-dimensional mold. The at least one plasticizer may comprise glycerin present in an amount ranging from 5 g to 10 g. The at least one plasticizer may further comprise propylene glycol present in an amount ranging from 3 g to 6 g. The method may further comprise adding xanthan gum in an amount ranging from 0.5 g to 1 g while stirring. The dehydrating may comprise air drying for 6 to 10 hours using the three-dimensional mold configured to expose one surface of the molded solution to air. The exposed surface may become the first surface having greater tackiness and a surface in contact with the mold may become the second surface having greater structural stability.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF FIGURES

The description will be more fully understood with reference to the following figures, which are presented as embodiments of the invention and should not be construed as a complete recitation of the scope of the invention, wherein:

FIG. 1 illustrates a cross-sectional view of a two-layer biopolymer structure, according to aspects of the present disclosure.

FIG. 2 illustrates a cross-sectional view of a three-layer biopolymer structure, according to aspects of the present disclosure.

FIG. 3 illustrates a cross-sectional view of a bioplastic layer structure, according to aspects of the present disclosure.

FIG. 4 illustrates a flowchart of a basic manufacturing process for a biopolymer composition, according to aspects of the present disclosure.

FIG. 5 illustrates a flowchart of a polyol hybrid manufacturing process for a biopolymer composition, according to aspects of the present disclosure.

FIG. 6 illustrates a flowchart of a multi-layer fabrication process for a biopolymer structure, according to aspects of the present disclosure.

FIG. 7 illustrates a flowchart of a product usage and lifecycle process, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

It will be understood that the components of the embodiments, as generally described herein and illustrated in the appended figures, may be arranged and designed in a variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages and similar language throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment. Thus, the phrases “in one embodiment,” “in an embodiment,” and similar language throughout this specification may but do not necessarily, all refer to the same embodiment.

The present disclosure relates to sustainable biopolymer compositions for skin-contact applications. The sustainable biopolymer compositions described herein may provide biodegradable alternatives to conventional synthetic materials used in products including, functional products, such as de-stress squishy toys; skincare applications, such as, anti-aging (retinol, peptides), collagen boosters, skin brightening, anti-wrinkle, vitamin infusion, hydration masks, invisible pore smoothing, temporary wrinkle filling, skin tightening, contouring patches, sweat-blocking patches, fragrance-emitting patches, skin whitening patches, bandages, pimple patches, eye patches, anti-chafing patches, cooling patches, and heel protection devices; and decorative and fashion applications, such as, nipple covers, jewelry-like skin patches, LED skin displays, temporary tattoos, silicone nose prosthetics, UV-reactive fashion materials, and creative 3D silicone prosthetics.

In some cases, a sustainable biopolymer composition may comprise a biodegradable polymer matrix formed from one or more naturally-derived polymers. The biodegradable polymer matrix may include polysaccharide-based polymers, plant-derived polymers, microbial polysaccharides, animal-derived polymers or combinations thereof. Embodiments of the polymer gelling agents may form stable, thermally gelling networks. Embodiments of the polymer gelling agents may include thermo-responsive protein networks that provide elasticity to the resulting material. The selection of the biodegradable polymer may influence properties such as elasticity, gel firmness, and thermal responsiveness of the resulting material. Embodiments incorporating natural polysaccharides may include, for example, carrageenan (kappa, iota, lambda), alginate (sodium alginate, calcium alginate), fucoidan, and ulvan, among others, and combinations thereof. Embodiments incorporating plant-derived polymers may include, for example, agar, pectin, cellulose derivatives, such as, CMC (carboxymethyl cellulose), HPMC (hydroxypropyl methylcellulose), HPC, EC (ethyl cellulose), guar gum, locust bean gum, xanthan gum, konjac glucomannan, tara gum, psyllium husk polysaccharide, starch (native or modified), pullulan, and inulin, among others, and combinations thereof. Embodiments incorporating microbial polysaccharides may include, for example, gellan gum, dextran, levan, and bacterial cellulose, among others, and combinations thereof. Embodiments incorporating animal-derived polymers may include, for example, gelatin, collagen, chitosan, hyaluronic acid and fibrin, among others, and combinations thereof. Embodiments may also include other alternatives to synthetic or semi-synthetic polymers, including, for example, polyvinyl alcohol (PVA), polyethylene glycol (PEG), polyacrylic acid, polyacrylamide, carbomer, polyurethane hydrogels, silicone elastomers, polycaprolactone (PCL), PLA/PLGA, polyvinylpyrrolidone (PVP), and thermoplastic elastomers, among others, and combination thereof.

The biodegradable polymer matrix may further include one or more humectants or plasticizers. Humectants and plasticizers may modulate properties such as hydration, flexibility, tack, and softness of the material. In some cases, the humectant or plasticizer may comprise a sugar, including, disaccharides, monosaccharides, sugar alcohols (polyols), and oligosaccharides, among others. Embodiments incorporating disaccharides may include, for example, lactose, maltose, trehalose and isomaltulose, among others, and combinations thereof. Embodiments incorporating monosaccharides may include, for example, glucose, fructose, galactose, mannose, ribose and xylose, among others, and combinations thereof. Embodiments incorporating sugar alcohols may include, for example, sorbitol, mannitol, xylitol, erythritol, maltitol, isomalt, glycerol and propylene glycol, among others, and combinations thereof. Embodiments incorporating oligosaccharides may include, for example, maltodextrin, fructooligosaccharides (FOS) and galactooligosaccharides (GOS), among others, and combinations thereof. Alternatives to traditional plasticizers may also be used in embodiments including: water content variation and ionic liquids (particularly biocompatible ionic liquids). The incorporation of humectants or plasticizers may reduce crystallization and tune the mechanical properties of the biodegradable polymer matrix.

In some cases, the sustainable biopolymer composition may include one or more functional additives. Functional additives may be incorporated into the biodegradable polymer matrix to impart additional properties to the material. The functional additives may be introduced into the polymer matrix in liquid form prior to a dehydration or curing process.

The sustainable biopolymer composition may be configured for direct application to skin or for incorporation into garments and wearable products. In some cases, the material may function as an adhesive layer applied to a backing substrate. In other cases, the material may be molded into a self-supporting structure having a shape corresponding to a desired end-use application. The material may exhibit skin-friendly properties and may be configured for repeated use prior to disposal.

The sustainable biopolymer composition may be biodegradable and may be disposed of through dissolution in water or through standard waste disposal methods. The biodegradable nature of the material may provide environmental benefits compared to conventional synthetic materials used in similar applications.

The biodegradable polymer matrix may incorporate one or more functional additives to impart additional properties to the resulting material. Functional additives may be introduced into the liquid formulation prior to dehydration or curing processes. The incorporation of functional additives may enable the creation of multifunctional patches and skin-contact products with properties beyond adhesion and structural support.

In some cases, fragrance compounds may be added to the liquid formulation before dehydration. The fragrance compounds may be dispersed throughout the biodegradable polymer matrix during the mixing and heating stages of formulation. Upon dehydration, the fragrance compounds may become entrapped within the solidified polymer network. The resulting material may function as a solid perfume that releases fragrance upon contact with skin. The incorporation of scents into the liquid formulation prior to dehydration may produce multifunctional patches that provide both adhesive functionality and fragrance delivery.

In some cases, nourishing oils may be added to the biodegradable polymer matrix. Nourishing oils may include plant-derived oils or other lipid-based compounds that provide moisturizing or conditioning effects to skin. The incorporation of nourishing oils may create nourishing effects after wearing the material on skin. The nourishing oils may prevent formation of red ring marks on skin from prolonged wear. The red ring marks may result from localized irritation or pressure at the edges of adhesive patches, and the nourishing oils may mitigate such effects through moisturization and skin conditioning.

In some cases, menthol may be added as a cooling agent into the material in liquid form. Menthol may be incorporated into the biodegradable polymer matrix during the liquid phase of processing. The menthol may become distributed throughout the biodegradable polymer matrix upon solidification. The resulting material may exhibit cooling functionality when applied to skin. Cooling patches incorporating menthol may be suitable for applications such as athletic recovery, post-exercise cooling, or therapeutic cooling for patients during recovery periods.

In some cases, mint essential oils may be added to the biodegradable polymer matrix. Mint essential oils may be configured to provide cooling sensations, fragrance, or antimicrobial properties. The mint essential oils may interact with thermoreceptors in the skin to produce cooling sensations, and the polymer matrix may modulate the rate of mint essential oil release to provide prolonged cooling effects.

In some cases, potassium preservatives may be incorporated into the biodegradable polymer matrix. Potassium preservatives may be configured to extend shelf life and prevent microbial growth during storage. The potassium preservatives may inhibit the growth of bacteria, fungi, or other microorganisms that could degrade the material or cause adverse effects upon skin contact.

In some cases, biosensors may be incorporated into the biodegradable polymer matrix to create smart patches with sensing capabilities. Biosensors may include electrochemical sensors, optical sensors, or other sensing elements configured to detect physiological parameters or biomarkers. The biosensors may be embedded within or attached to the biodegradable polymer matrix during fabrication. Smart patches incorporating biosensors may enable monitoring of skin conditions, hydration levels, temperature, or other parameters relevant to health and wellness applications. The combination of biodegradable materials with biosensor technology may provide disposable or limited-use sensing platforms with reduced environmental impact compared to conventional sensor substrates.

In some cases, pyrrolidone carboxylic acid (PCA) or a salt thereof may be added to the biodegradable polymer matrix to enhance hydration behavior. PCA may be incorporated in a range of 0.05 wt % to 15 wt %. PCA may function as an osmolyte that provides skin-native hydration behavior to the resulting material. The PCA or salt thereof may be introduced alongside sucrose to enable combined hydration control and reduced residue formation on the skin surface. PCA-type osmolytes may be blended with sucrose within the polymer network to retain the functional effects of sucrose while gaining hydration properties that are compatible with skin physiology.

In some cases, bioadhesive polymer components may be incorporated into the biodegradable polymer matrix to increase affinity for skin. Bioadhesive polymer components may include chitosan derivatives or carboxyl-containing polymers. Chitosan derivatives may be introduced into the base matrix to enhance adhesion to skin surfaces. Carboxyl-containing polymers may similarly be incorporated or grafted into the base matrix to increase bioadhesive functionality. The bioadhesive polymer component may be balanced with humectant content to support adhesion without causing skin irritation. The combination of bioadhesive polymer components with the base formulations may provide enhanced skin contact and retention compared to formulations lacking bioadhesive augmentation.

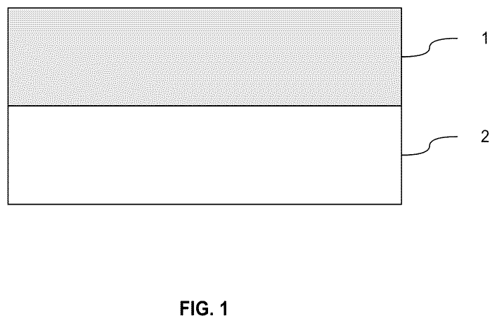

Referring to FIG. 1, a two-layer sustainable biopolymer article for skin-contact applications is shown. The two-layer structure includes a structural-backing layer 1 and a skin contact layer 2. The structural-backing layer 1 may be positioned above the skin contact layer 2 in the illustrated configuration.

The structural-backing layer 1 may comprise a dried agar-sucrose formulation. The dried agar-sucrose formulation of the structural-backing layer 1 may provide structural support and dimensional stability to the two-layer article. The structural-backing layer 1 may utilize a baseline agar-sucrose formulation that has been processed to achieve a dried state. The dried state of the structural-backing layer 1 may maintain the overall shape of the two-layer structure during use.

With continued reference to FIG. 1, the skin contact layer 2 may comprise a softer agar-sucrose formulation. The softer agar-sucrose formulation of the skin contact layer 2 may incorporate a higher glycerin content compared to the structural-backing layer 1. The glycerin content in the skin contact layer 2 may be in a range of 10 g to 20 g. The higher glycerin content in the skin contact layer 2 may increase tack and conformability of the skin contact layer 2. The skin contact layer 2 may be configured to contact skin and provide adhesion to skin surfaces. The enhanced softness of the skin contact layer 2 may improve adhesion to skin surfaces while the structural-backing layer 1 provides mechanical support.

The fabrication of the two-layer structure may involve a sequential process. The structural-backing layer 1 may be formed first and allowed to partially dry. When the surface of the structural-backing layer 1 becomes partially dried, the skin contact layer 2 may be poured onto the structural-backing layer 1. An interface between the structural-backing layer 1 and the skin contact layer 2 may form during the sequential fabrication process. Polymer chains of the skin contact layer 2 may interpenetrate with the surface of the structural-backing layer 1 during gelation. The interpenetration of polymer chains may create a cohesive bond between the structural-backing layer 1 and the skin contact layer 2. The cohesive bond may resist delamination during use of the two-layer article.

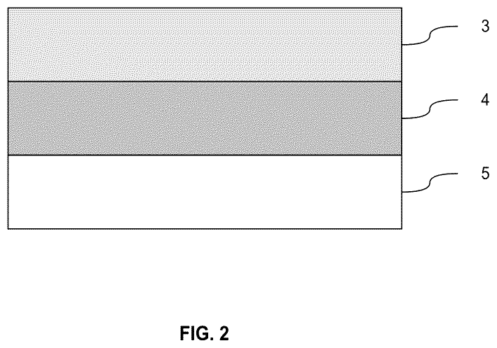

Referring to FIG. 2, a three-layer fabric composite structure for skin-contact applications is shown. The three-layer structure includes a starch-based cover layer 3, a fabric layer 4, and an agar-sucrose-based insert 5. The starch-based cover layer 3 may be positioned above the fabric layer 4, and the fabric layer 4 may be positioned above the agar-sucrose-based insert 5 in the illustrated configuration.

The starch-based cover layer 3 may comprise tapioca starch combined with agar and sucrose. The tapioca starch may be present in a range of 2 g to 10 g. The starch-based cover layer 3 may incorporate the baseline level of agar and sucrose along with the tapioca starch. The starch-based cover layer 3 may serve as an outer cover that imparts durability and structural integrity to the three-layer fabric composite structure. The combination of tapioca starch with agar and sucrose in the starch-based cover layer 3 may provide a durable outer surface that protects the underlying layers from mechanical damage and environmental exposure.

With continued reference to FIG. 2, the fabric layer 4 may comprise a transparent fabric. The transparent fabric of the fabric layer 4 may comprise nylon tulle, power mesh, spandex mesh, or pantyhose material. The fabric layer 4 may increase stretchability of the three-layer fabric composite structure while maintaining a low-profile appearance. The transparent fabric of the fabric layer 4 may reinforce the composite structure and may impart stretchability that allows the material to accommodate body movement without restricting motion. The pantyhose material may serve as a transparent fabric option for the fabric layer 4.

The agar-sucrose-based insert 5 may be positioned at the bottom of the three-layer fabric composite structure. The agar-sucrose-based insert 5 may provide adhesion and skin contact functionality to the three-layer fabric composite structure. The agar-sucrose-based insert 5 may be configured to contact skin and secure the three-layer fabric composite structure to the body.

The fabrication of the three-layer fabric composite structure may involve a sequential process. The starch-based cover layer 3 may be created first as the outermost layer. When the starch-based cover layer 3 has dried and exhibits no stickiness, the fabric layer 4 may be inserted onto the starch-based cover layer 3. The agar-sucrose-based insert 5 may then be added onto the fabric layer 4 to complete the three-layer fabric composite structure. The sequential fabrication process may create a laminated structure in which each layer contributes distinct functional properties to the overall composite. The combination of the starch-based cover layer 3, the fabric layer 4, and the agar-sucrose-based insert 5 may distribute mechanical stresses across the composite structure, with the fabric layer 4 bearing tensile loads while the starch-based cover layer 3 and the agar-sucrose-based insert 5 provide durability and adhesion respectively.

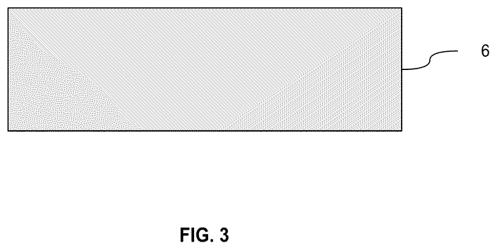

Referring to FIG. 3, a bioplastic layer structure for skin-contact applications is shown. The bioplastic layer structure includes a bioplastic layer 6. The bioplastic layer 6 may be incorporated into multi-layer structures to provide additional structural properties or barrier characteristics.

The bioplastic layer 6 may comprise starch combined with an organic acid. Although any suitable starch content may be included, in various embodiments the starch content of the bioplastic layer 6 may be in a range of 2 g to 10 g. The organic acid may comprise citric acid, and although any suitable organic acid concentration may be used in some embodiments an organic acid concentration in a range of 3 g to 5 g is used. The combination of starch and citric acid may form a bioplastic material that provides structural properties to the bioplastic layer 6.

With continued reference to FIG. 3, the bioplastic layer 6 may comprise fruit acid as an alternative to citric acid. Although any suitable fruit acid concentration may be used, in some embodiments the fruit acid may be present in a range of 3 g to 5 g. The fruit acid may function similarly to citric acid in forming the bioplastic material of the bioplastic layer 6. The selection between citric acid and fruit acid may depend on desired material properties or formulation requirements.

The bioplastic layer 6 may be added to multi-layer configurations to enhance durability, moisture resistance, or other functional properties of the resulting composite material. The bioplastic layer 6 may provide barrier characteristics that protect underlying layers or skin surfaces from environmental exposure. The starch and acid-based composition of the bioplastic layer 6 may maintain biodegradability while imparting structural reinforcement to the multi-layer article.

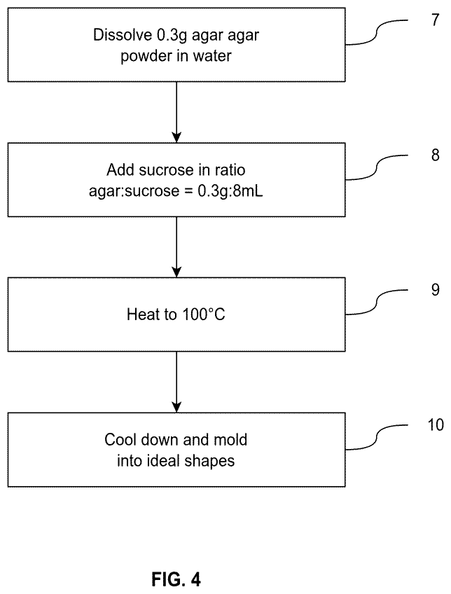

Referring to FIG. 4, a basic manufacturing process for preparing an agar-sucrose-based biopolymer composition is shown. The basic manufacturing process includes a step 7, a step 8, a step 9, and a step 10. The steps 7, 8, 9, and 10 may be performed sequentially to produce the sustainable biopolymer material.

The step 7 comprises dissolving agar powder in water. Although any suitable gelling agent concentration may be used in some embodiments the gelling agent may comprise agar present in an amount ranging from 0.3 g to 0.9 g. The agar may be added to water and mixed to initiate dissolution of the agar powder. The dissolution of the agar in water may form a solution that serves as the basis for the biodegradable polymer matrix.

With continued reference to FIG. 4, the step 8 comprises adding sucrose to the solution. Although any suitable concentration of sucrose may be used, in some embodiments the sucrose may be present in an amount ranging from 5 mL to 12 mL. Although any suitable ratio of gelling agent to sucrose may be used, in some embodiments the gelling agent may comprise agar in a ratio of approximately 0.3 g agar to 8 mL sucrose. The sucrose may be added to the solution after the agar has been dissolved in the water. The sucrose may function as a humectant and plasticizer within the polymer matrix. The addition of sucrose may modulate gel firmness, hydration, and tack within the agar matrix.

The step 9 comprises heating the mixture to approximately 100 degrees Celsius. The agar may be dissolved in water and heated to approximately 100 degrees Celsius to achieve dissolution and gelation. The heating process may facilitate formation of a clear solution. The heating to approximately 100 degrees Celsius may ensure complete dissolution of the agar and sucrose components within the water. The clear solution formed during the step 9 may indicate that the agar and sucrose have been fully incorporated into the aqueous phase.

As further shown in FIG. 4, the step 10 comprises cooling and molding the composition into desired shapes. The solution may be cooled from the elevated temperature of the step 9 to a temperature suitable for molding operations. The cooled solution may be poured into molds having shapes corresponding to desired end-use applications. The molding process may form the sustainable biopolymer material into configurations such as nipple covers, skin patches, or other skin-contact articles. Upon cooling within the molds, the agar-sucrose composition may gel and solidify into the desired shape. The molded composition may then be subjected to dehydration to form the final biodegradable adhesive material.

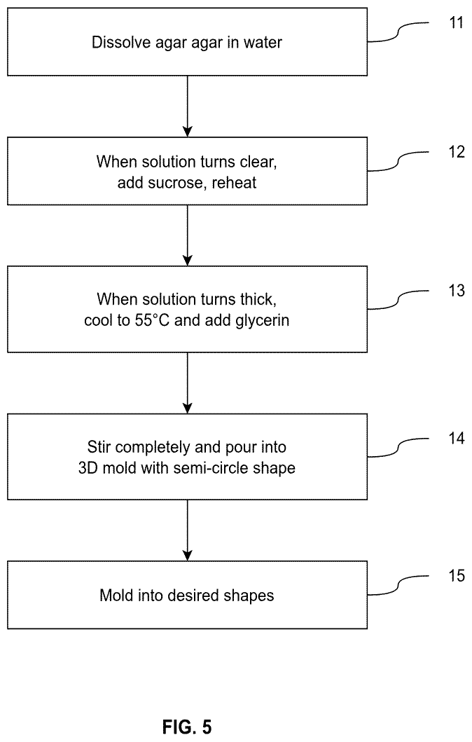

Referring to FIG. 5, a polyol hybrid manufacturing process for preparing a biopolymer composition with enhanced flexibility is shown. The polyol hybrid manufacturing process includes a step 11, a step 12, a step 13, a step 14, and a step 15. The steps 11, 12, 13, 14, and 15 may be performed sequentially to produce a sustainable biopolymer material with modified mechanical properties.

The step 11 comprises dissolving agar in water. The agar may be present in an amount ranging from 0.3 g to 0.9 g. The agar may be added to water and mixed to initiate dissolution. The dissolution of the agar in water may form a solution that serves as the basis for the biodegradable polymer matrix. The solution may be heated during the step 11 until the solution turns clear. The clear appearance of the solution may indicate that the agar has been fully dissolved in the aqueous phase.

With continued reference to FIG. 5, the step 12 comprises adding sucrose to the solution and reheating. The sucrose may be added to the solution when the solution turns clear. Although any suitable concentration of sucrose may be used, in some embodiments the sucrose may be present in an amount ranging from 5 mL to 12 mL. After adding the sucrose, the solution may be reheated. The reheating may continue until the solution turns thick. The thickened appearance of the solution may indicate that the sucrose has been incorporated into the agar matrix and that the solution is ready for the subsequent cooling and plasticizer addition steps.

The step 13 comprises cooling the solution and adding glycerin. The solution may be cooled to approximately 55 degrees Celsius when the solution turns thick. The controlled cooling temperature of approximately 55 degrees Celsius may facilitate uniform dispersion of plasticizers throughout the polymer matrix while maintaining the solution in a workable state for subsequent operations. Although any suitable concentration of glycerin may be used, in some embodiments glycerin may be added to the cooled solution in a range of 5 g to 10 g. The addition of glycerin may reduce crystallization risk associated with sucrose and may tune softness of the biodegradable polymer matrix. Glycerin may function synergistically with sucrose to modulate hydration and plasticization within the polymer network.

As further shown in FIG. 5, sorbitol may be added as an alternative polyol additive in the step 13. Sorbitol may reduce crystallization risk and tune softness when combined with sucrose and agar or gelatin. Sorbitol may provide similar plasticizing effects to glycerin and may be selected based on desired material properties or formulation requirements. The selection between glycerin and sorbitol may depend on the target mechanical characteristics of the resulting biopolymer material.

In some cases, propylene glycol may be added during the step 13. Although any suitable concentration of propylene glycol may be used, in some embodiments propylene glycol may be present in a range of 3 g to 6 g. Propylene glycol may function as an additional plasticizer that modulates flexibility and hydration properties of the biodegradable polymer matrix. Although any suitable concentration of xanthan gum may be used, in some embodiments xanthan gum may also be added during the step 13 in a range of 0.5 g to 1 g. Xanthan gum may function as a rheology modifier that influences viscosity and flow properties of the solution during processing. The combination of propylene glycol and xanthan gum may be added to the solution while stirring until the solution turns clear.

With continued reference to FIG. 5, the step 14 comprises stirring the solution and pouring the solution into a three-dimensional mold. The solution may be stirred completely to ensure uniform distribution of the plasticizers and polyols throughout the polymer matrix. The three-dimensional mold may have a semi-circular shape. The semi-circular shape of the three-dimensional mold may be configured to form any suitable product, including, for example, a nipple cover. The solution may be poured into the three-dimensional mold with the semi-circular shape after the stirring has been completed.

The step 15 comprises molding the composition into desired shapes. The solution within the three-dimensional mold may gel and solidify as the temperature decreases. The molding process may form the sustainable biopolymer material into configurations corresponding to the shape of the three-dimensional mold. The semi-circular shape may be suitable for applications such as covers, patches, or other skin-contact products. The molded composition may then be subjected to dehydration processes to form the final biodegradable adhesive material with enhanced flexibility from the incorporated polyols.

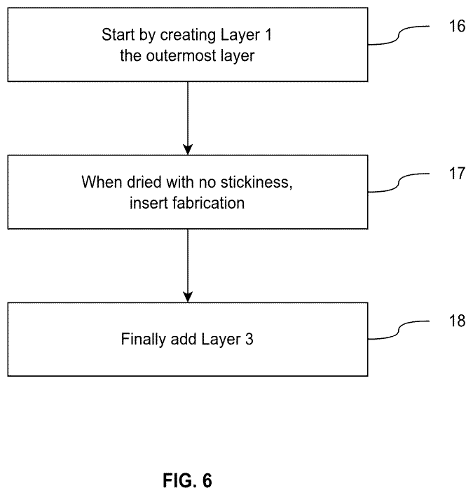

Referring to FIG. 6, a multi-layer fabrication process for creating a layered biopolymer structure is shown. The multi-layer fabrication process includes a step 16, a step 17, and a step 18. The steps 16, 17, and 18 may be performed sequentially to produce a multi-layer sustainable biopolymer article such as the three-layer fabric composite structure described with reference to FIG. 2.

The step 16 comprises creating the outermost layer first. The outermost layer may comprise the starch-based cover layer 3 described with reference to FIG. 2. The starch-based cover layer 3 may be formed by combining tapioca starch with agar and sucrose at baseline levels. The tapioca starch may be present in a range of 2 g to 10 g. The starch-based cover layer 3 may be poured into a mold and allowed to dry. The drying process may remove moisture from the starch-based cover layer 3 and may cause the starch-based cover layer 3 to solidify into a stable configuration. The starch-based cover layer 3 may serve as a durable outer cover that provides structural integrity to the resulting multi-layer article.

With continued reference to FIG. 6, the step 17 comprises inserting the fabric layer 4 when the outermost layer is dried with no stickiness. The starch-based cover layer 3 may be monitored during the drying process to determine when the surface exhibits no stickiness. The absence of stickiness on the surface of the starch-based cover layer 3 may indicate that the starch-based cover layer 3 has dried sufficiently to receive the fabric layer 4. The fabric layer 4 may comprise a transparent fabric such as nylon tulle, power mesh, spandex mesh, or pantyhose material. The fabric layer 4 may be positioned onto the dried surface of the starch-based cover layer 3. The fabric layer 4 may increase stretchability of the multi-layer article while maintaining a low-profile appearance.

The step 18 comprises adding the final agar-sucrose-based layer to complete the composite structure. The agar-sucrose-based insert 5 may be poured onto the fabric layer 4 after the fabric layer 4 has been positioned on the starch-based cover layer 3. The agar-sucrose-based insert 5 may comprise an agar-sucrose formulation that provides adhesion and skin contact functionality. The agar-sucrose-based insert 5 may flow around and through portions of the fabric layer 4 during the pouring process. The interpenetration of the agar-sucrose-based insert 5 with the fabric layer 4 may create a cohesive bond between the fabric layer 4 and the agar-sucrose-based insert 5. The agar-sucrose-based insert 5 may then be allowed to gel and solidify to complete the three-layer fabric composite structure. The sequential fabrication process of the steps 16, 17, and 18 may create a laminated structure in which the starch-based cover layer 3 provides durability, the fabric layer 4 provides stretchability, and the agar-sucrose-based insert 5 provides adhesion to skin surfaces.

Aa dehydration process for biopolymer compositions using three-dimensional molds is also provided. The dehydration process comprises air drying with three-dimensional molds for 6 to 10 hours. The air drying process may be conducted using three-dimensional molds that are configured to expose one surface of the molded solution to air during the drying process.

The three-dimensional molds may position the material such that one surface remains exposed to the surrounding atmosphere while an opposite surface contacts a mold surface. The exposed surface may experience faster evaporation and water-air exchange compared to the surface in contact with the mold. The differential drying rates between the two surfaces may result in asymmetric properties across the thickness of the material.

The exposed surface of the material during air drying may become more sticky due to faster evaporation and higher concentration of humectants and plasticizers at that surface. The faster moisture loss at the exposed surface may result in a higher concentration of humectants and plasticizers at that surface relative to the polymer content. The elevated humectant concentration at the exposed surface may increase tack and adhesive properties of that surface. The surface in contact with the mold may retain moisture longer during the drying process. The slower drying rate at the mold-contacting surface may allow the polymer network at that surface to consolidate into a more stable, less tacky configuration. The differential drying rates may produce a material having an adhesive surface configured for skin contact and a stable backing surface configured for handling and structural support.

The dehydration process may further include a heat thickening step prior to pouring the solution into the three-dimensional mold. The solution may be thickened by heating for 30 to 60 seconds prior to pouring into the mold. The heat thickening step may increase viscosity of the solution and may facilitate formation of a more concentrated polymer matrix. The thickened solution may be poured into the three-dimensional mold after the heat thickening step has been completed. The combination of heat thickening and air drying may produce a material having desired mechanical and adhesive properties. The asymmetric surface properties may be achieved without the need for separate adhesive coatings or backing materials, simplifying the manufacturing process and maintaining the fully biodegradable composition of the material.

Various product forms that may be molded using the biopolymer compositions are shown. The product forms may include, for example, functional products, such as de-stress squishy toys; skincare applications, such as, anti-aging (retinol, peptides), collagen boosters, skin brightening, anti-wrinkle, vitamin infusion, hydration masks, invisible pore smoothing, temporary wrinkle filling, skin tightening, contouring patches, sweat-blocking patches, fragrance-emitting patches, skin whitening patches, bandages, pimple patches, eye patches, anti-chafing patches, cooling patches, and heel protection devices; and decorative and fashion applications, such as, nipple covers, jewelry-like skin patches, LED skin displays, temporary tattoos, silicone nose prosthetics, UV-reactive fashion materials, and creative 3D silicone prosthetics. Each product form may be manufactured using the sustainable biopolymer compositions and manufacturing processes described herein.

The nipple cover may be formed using semi-circular molds. The semi-circular shape of the nipple cover may be configured to conform to the contours of the body. The nipple cover may provide coverage and support while exhibiting skin-friendly adhesion properties. The nipple cover may be manufactured using the three-dimensional molds with semi-circular shapes. The biodegradable polymer matrix of the nipple cover may conform to body contours and may be reused multiple times prior to disposal.

The skin patch may be configured for general skin-contact applications. The skin patch may be applied to various areas of the body for adhesive functionality, delivery of functional additives, or protective barrier applications. The skin patch may incorporate the sustainable biopolymer compositions described herein to provide biodegradable adhesion to skin surfaces. The skin patch may serve as a platform for incorporating fragrance compounds, nourishing oils, or other functional additives into the biodegradable polymer matrix.

The anti-chafing patch may be configured for athletes. The anti-chafing patch may be applied to areas of the body prone to friction during athletic activity. Friction-prone areas may include inner thighs, underarms, or areas beneath sports bras or athletic garments. The anti-chafing patch may reduce friction between skin surfaces or between skin and clothing during movement. The biodegradable polymer matrix of the anti-chafing patch may provide a protective barrier that prevents chafing while maintaining breathability and comfort during athletic performance. The flexibility of the anti-chafing patch may allow the anti-chafing patch to move with the body without restricting motion or causing discomfort.

The eye patch may be formed in shapes configured to conform to the contours of the under-eye area or the area surrounding the eye. The eye patch may be configured for cosmetic or therapeutic use. Cosmetic applications of the eye patch may include delivery of moisturizing or conditioning ingredients to the delicate skin around the eyes. Therapeutic applications of the eye patch may include protection, hydration, or delivery of medicinal compounds to the eye area. The shape of the eye patch may accommodate the curved contours of the under-eye region to maintain contact with the skin surface during wear.

The pimple patch may be configured for acne treatment applications. The pimple patch may be formed in small, discrete shapes configured for application over individual blemishes or acne lesions. The pimple patch may be configured to absorb exudate from blemishes while protecting the affected area from external contamination. The biodegradable polymer matrix of the pimple patch may maintain a moist environment at the skin surface while absorbing exudate from the blemish. The pimple patch may incorporate pimple treatment actives that reduce inflammation or deliver antimicrobial agents to the affected skin. The adhesion properties of the pimple patch may secure the pimple patch in place over treatment areas while the biodegradable composition may allow for comfortable removal without leaving residue.

The cooling patch may incorporate menthol for cooling functionality. The cooling patch may utilize the polymer matrix as a carrier for menthol or other cooling agents. The menthol within the cooling patch may interact with thermoreceptors in the skin to produce cooling sensations. The hydrogel structure of the cooling patch may provide sustained release of the cooling compounds to the skin surface. The cooling patch may be applied to the forehead, neck, or other body areas to provide relief from heat or discomfort. The cooling patch may be suitable for applications such as athletic recovery or post-exercise cooling.

The cooling patch for recovery may be configured for cancer patients experiencing discomfort, inflammation, or elevated skin temperature as a result of cancer treatments. The cooling patch for recovery may provide therapeutic cooling to patients during recovery periods. The skin-friendly and biodegradable properties of the cooling patch for recovery may be suitable for patients with sensitive skin or compromised skin barrier function. The cooling patch for recovery may incorporate menthol or other cooling agents within the biodegradable polymer matrix to provide a cooling sensation upon application to skin. The sustained release of cooling compounds from the cooling patch for recovery may enable extended wear without irritation.

In many embodiments an adhesive material or product for skin-contact applications may be provided. The adhesive material may be configured as a bandage adhesive backing. The adhesive material may be applied to the back of pimple patches as a backing adhesive layer. The adhesive material may provide skin-friendly adhesion that secures the pimple patch to the skin surface while allowing active ingredients of the pimple patch to contact the affected area. The biodegradable nature of the adhesive material may provide environmental benefits compared to conventional synthetic adhesives used in pimple patch products.

The adhesive material may be applied to the back of bandages as a backing adhesive layer. The adhesive material may secure the bandage to the skin surface surrounding a wound or injury site. The skin-friendly properties of the adhesive material may reduce irritation during wear and removal of the bandage. The adhesive material may maintain a moist wound environment that supports the natural healing process when applied as a bandage adhesive backing.

The adhesive material may be applied to the back of skin patches as a backing adhesive layer. Skin patches may include transdermal drug delivery patches, cosmetic patches, or other patch-type products designed for application to skin. The adhesive material may provide secure attachment to the skin surface while maintaining compatibility with active components of the skin patch. The biodegradable polymer matrix of the adhesive material may function as a reservoir that controls delivery rate of functional additives to the skin surface.

A heel protection adhesive may be configured for heel protection applications. The heel protection adhesive may be applied within footwear to improve fit of shoes around the heel area. The heel protection adhesive may prevent slippage of the heel within the shoe. The prevention of heel slippage may reduce the occurrence of blisters or discomfort associated with poorly fitting footwear. The heel protection adhesive may cushion the heel and reduce friction between the heel and the interior surface of shoes. The biodegradable polymer matrix of the heel protection adhesive may conform to the contours of the heel area and may maintain adhesion during walking or other activities.

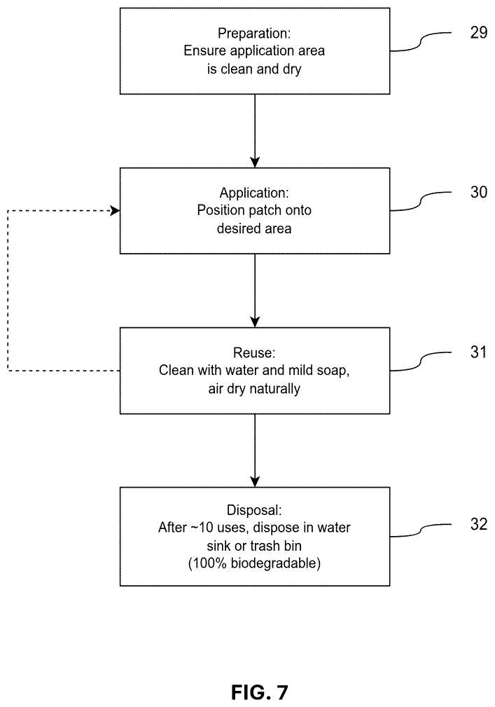

Referring to FIG. 7, a product usage and lifecycle process for biopolymer skin-contact articles is shown. The product usage and lifecycle process includes a preparation step 29, an application step 30, a reuse step 31, and a disposal step 32. The preparation step 29, the application step 30, the reuse step 31, and the disposal step 32 may be performed to use, maintain, and dispose of the sustainable biopolymer articles described herein, such as functional products, such as de-stress squishy toys; skincare applications, such as, anti-aging (retinol, peptides), collagen boosters, skin brightening, anti-wrinkle, vitamin infusion, hydration masks, invisible pore smoothing, temporary wrinkle filling, skin tightening, contouring patches, sweat-blocking patches, fragrance-emitting patches, skin whitening patches, bandages, pimple patches, eye patches, anti-chafing patches, cooling patches, and heel protection devices; and decorative and fashion applications, such as, nipple covers, jewelry-like skin patches, LED skin displays, temporary tattoos, silicone nose prosthetics, UV-reactive fashion materials, and creative 3D silicone prosthetics.

The preparation step 29 comprises ensuring the application area is clean and dry. A user may clean the skin application area prior to positioning the sustainable biopolymer article onto the skin. The cleaning of the skin application area may remove skin oils, perspiration, dust, or other contaminants that could interfere with adhesion of the sustainable biopolymer article. The skin application area may be dried after cleaning to provide a dry surface for application. The clean and dry skin surface may promote adhesion between the sustainable biopolymer article and the skin. The preparation step 29 may be performed each time the sustainable biopolymer article is applied to the skin, including during initial use and during subsequent reuse cycles.

With continued reference to FIG. 10, the application step 30 comprises positioning the sustainable biopolymer article onto the skin application area. The sustainable biopolymer article may be positioned such that the skin contact layer 2 or the agar-sucrose-based insert 5 contacts the skin surface. The user may press the sustainable biopolymer article against the skin to secure adhesion. The pressing of the sustainable biopolymer article against the skin may increase contact area between the adhesive surface and the skin surface. The increased contact area may enhance adhesive bonding through van der Waals interactions and mechanical interlocking with skin surface features. The sustainable biopolymer article may conform to the contours of the body during the application step 30.

The reuse step 31 comprises cleaning the sustainable biopolymer article with water and mild soap followed by air drying. After the sustainable biopolymer article has been removed from the skin following use, the sustainable biopolymer article may be washed with water and mild soap. The water and mild soap may remove skin oils, perspiration, dust, or other contaminants that accumulate on the sustainable biopolymer article during wear. The mild soap may be selected to avoid harsh detergents or chemicals that could degrade the biodegradable polymer matrix or alter the adhesive properties of the sustainable biopolymer article. After washing with water and mild soap, the sustainable biopolymer article may be allowed to air dry naturally. The air drying process may restore the adhesive properties of the sustainable biopolymer article and prepare the sustainable biopolymer article for subsequent use. A dashed arrow in FIG. 10 indicates the reuse cycle returning from the reuse step 31 back to the application step 30 for repeated use. The cleaning and air drying cycle of the reuse step 31 may be repeated after each use. The sustainable biopolymer article may be configured for approximately 10 uses before disposal. The reusability of the sustainable biopolymer article may provide economic and environmental benefits compared to single-use products.

As further shown in FIG. 10, the disposal step 32 comprises disposing the sustainable biopolymer article after approximately 10 uses. The sustainable biopolymer article may be biodegradable and configured for disposal in water under running water or in a waste receptacle. In some cases, the sustainable biopolymer article may be disposed of in a water sink under running water. The running water may dissolve or break down the biodegradable polymer matrix, allowing the sustainable biopolymer article to be washed away through standard plumbing systems. The dissolution of the sustainable biopolymer article in water may occur without leaving harmful residues or contributing to environmental pollution. In some cases, the sustainable biopolymer article may be disposed of in a waste receptacle such as a trash bin. The biodegradable polymer matrix may break down through natural decomposition processes when disposed of in standard waste streams. The biodegradable composition of the sustainable biopolymer article may allow the sustainable biopolymer article to decompose in landfill environments or composting facilities without persisting in the environment as synthetic materials would. The biodegradable disposal options of the disposal step 32 may reduce environmental impact associated with disposal of skin-contact products and may provide an environmentally responsible end-of-life pathway for the sustainable biopolymer article.

The sustainable biopolymer material may be configured as a therapeutic skin patch for wound care and healing applications. A therapeutic skin patch may be applied to wounds, abrasions, or other skin injuries to promote healing. The biodegradable polymer matrix of the therapeutic skin patch may maintain a moist wound environment that supports the natural healing process. The hydrogel properties of the polymer matrix may absorb exudate from the wound site while maintaining hydration at the wound surface. The moist wound environment created by the therapeutic skin patch may facilitate cell migration and tissue regeneration at the wound site. The therapeutic skin patch may also deliver therapeutic agents to the wound site to accelerate healing or prevent infection. The skin-friendly adhesion properties of the therapeutic skin patch may secure the therapeutic skin patch in place over the wound area without causing irritation to surrounding healthy skin. The biodegradable composition of the therapeutic skin patch may allow for comfortable removal without leaving residue on the wound site or surrounding skin. The therapeutic skin patch may be configured for single use or for limited reuse depending on the wound care application and the condition of the wound.

The sustainable biopolymer material may be configured as an odor-control patch for self-care applications. An odor-control patch may be applied to areas of the body prone to perspiration or odor generation. The biodegradable polymer matrix of the odor-control patch may incorporate fragrance compounds that reduce or mask body odor. The fragrance compounds may be dispersed throughout the biodegradable polymer matrix during formulation and may be released gradually as the odor-control patch contacts skin and as moisture migrates through the polymer network. The controlled release of fragrance compounds may provide sustained scent delivery throughout the wear period of the odor-control patch. In some cases, the odor-control patch may incorporate odor-neutralizing agents as an alternative to or in combination with fragrance compounds. The odor-neutralizing agents may chemically interact with odor-causing compounds to neutralize or reduce body odor rather than masking the odor with fragrance. The odor-control patch may be applied to the underarm area, feet, or other body regions as part of personal hygiene routines. The skin-friendly properties of the odor-control patch may allow for extended wear without causing irritation. The biodegradable composition of the odor-control patch may provide an environmentally responsible alternative to conventional odor-control products that contain synthetic materials.

The base polymer formulations, additives, multi-layer structures, and manufacturing processes described herein may interact synergistically to achieve the functional properties of the sustainable biopolymer material for activewear, undergarments, and skin-contact applications.

The biodegradable polymer matrix may provide structural integrity to the sustainable biopolymer material through formation of a gelling network. When agar or gelatin is dissolved in water and heated, the polymer chains may disperse throughout the solution. Upon cooling, the polymer chains may associate and form a three-dimensional network structure that provides mechanical strength and dimensional stability to the resulting material. The gelling network may entrap water molecules and other formulation components within the polymer matrix, creating a hydrogel structure that maintains cohesion while remaining flexible. The structural integrity provided by the polymer matrix may enable the material to maintain shape during wear and to resist tearing or fragmentation during application and removal from skin surfaces.

The plasticizers and polyols incorporated into the biodegradable polymer matrix may tune flexibility and adhesion characteristics of the material by disrupting intermolecular interactions within the polymer network. Sucrose, glycerol, sorbitol, and propylene glycol may insert between polymer chains and reduce the density of chain-to-chain associations within the gelling network. The reduction in intermolecular interactions may increase mobility of polymer chain segments and may lower the glass transition temperature of the material. The increased chain mobility may manifest as enhanced flexibility, allowing the material to conform to curved body surfaces and to accommodate movement during wear without cracking or delaminating. The plasticizers and polyols may also modulate tack and adhesion properties of the material by influencing surface energy and deformability of the polymer matrix. A more plasticized matrix may deform more readily upon contact with skin, increasing contact area between the material and the skin surface and enhancing adhesive bonding through van der Waals interactions and mechanical interlocking with skin surface features.

The humectant properties of sucrose, glycerol, and other polyols may contribute to the adhesion mechanism by maintaining moisture within the polymer matrix. The humectants may attract and retain water molecules from the surrounding environment, including moisture present on the skin surface. The retained moisture may plasticize the polymer matrix at the skin-material interface and may facilitate intimate contact between the material and the skin. The moisture at the interface may also contribute to adhesion through capillary forces and hydrogen bonding between the hydrated polymer matrix and the skin surface. The balance between humectant content and polymer network density may determine the equilibrium moisture content of the material and may influence adhesion strength and ease of removal from skin.

The dehydration process using three-dimensional molds may create differential stickiness on opposing surfaces of the material through asymmetric moisture removal. During air drying, the surface of the material exposed to the atmosphere may lose moisture more rapidly than the surface in contact with the mold. The faster moisture loss at the exposed surface may result in a higher concentration of humectants and plasticizers at that surface relative to the polymer content. The elevated humectant concentration at the exposed surface may increase tack and adhesive properties of that surface. Conversely, the surface in contact with the mold may retain moisture longer during the drying process, and the slower drying rate may allow the polymer network at that surface to consolidate into a more stable, less tacky configuration. The differential drying rates may thus produce a material having an adhesive surface configured for skin contact and a stable backing surface configured for handling and structural support. The asymmetric surface properties may be achieved without the need for separate adhesive coatings or backing materials, simplifying the manufacturing process and maintaining the fully biodegradable composition of the material.

The multi-layer structures described herein may leverage interactions between different formulation compositions to achieve enhanced functional performance. In a two-layer structure, a dried agar-sucrose formulation of a structural backing layer may provide a stable backing that resists deformation and maintains the overall shape of the product. A higher glycerin content in a skin contact layer may increase softness and tack of the skin contact layer, enhancing conformability to skin contours and improving adhesion strength. An interface between the structural backing layer and the skin contact layer may form during sequential fabrication when the skin contact layer is poured onto a partially dried surface of the structural backing layer. Polymer chains of the skin contact layer may interpenetrate with the surface of the structural backing layer during gelation, creating a cohesive bond between the two layers that resists delamination during use.

In a three-layer fabric composite structure, a starch-based layer may provide a durable outer surface that protects underlying layers from mechanical damage and environmental exposure. A transparent fabric layer may reinforce the composite structure and may impart stretchability that allows the material to accommodate body movement without restricting motion. An agar-sucrose-based layer may provide adhesive skin contact functionality that secures the composite to the body. The combination of the three layers may distribute mechanical stresses across the composite structure, with the fabric layer bearing tensile loads while the polymer layers provide adhesion and cushioning. The multi-layer configuration may enable creation of products that combine skin-friendly adhesion of the biodegradable polymer matrix with mechanical durability and stretchability of fabric reinforcement.

The functional additives incorporated into the biodegradable polymer matrix may interact with the polymer network and the skin surface to deliver additional benefits beyond adhesion and structural support. Fragrance compounds dispersed within the polymer matrix may be released gradually as the material contacts skin and as moisture migrates through the polymer network. The controlled release of fragrance may provide sustained scent delivery throughout the wear period. Menthol and other cooling agents may interact with thermoreceptors in the skin to produce cooling sensations, and the polymer matrix may modulate the rate of menthol release to provide prolonged cooling effects. Nourishing oils and skin care actives may migrate from the polymer matrix to the skin surface during wear, delivering moisturizing and conditioning benefits while the material remains in contact with skin. The polymer matrix may function as a reservoir that controls delivery rate of functional additives to the skin surface.

The interaction between the biodegradable polymer matrix and skin may be influenced by moisture dynamics at the skin-material interface. The humectants within the polymer matrix may draw moisture from the skin surface, which may enhance adhesion but may also affect skin hydration over extended wear periods. The incorporation of nourishing oils and skin care actives may counteract potential drying effects by delivering moisturizing compounds to the skin. The balance between humectant content and nourishing additive content may be adjusted to optimize both adhesion performance and skin compatibility for specific applications and wear durations.

A number of implementations have been described. Nevertheless, it will be understood that various modifications may be made without departing from the spirit and scope of the disclosure. Accordingly, other implementations are within the scope of the following claims.

### DOCTRINE OF EQUIVALENTS

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

As used herein, the singular terms “a,” “an,” and “the,” may include plural referents unless the context clearly dictates otherwise. Reference to an object in the singular is not intended to mean “one and only one” unless explicitly so stated, but rather “one or more.”

As used herein, the terms “approximately” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1 %, less than or equal to ±0.5%, less than or equal to ±0.1 %, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. Where ranges are described, the range should be understood to include the endpoints of the ranges, and the endpoints of such ranges are also contemplated to stand on their own as inventive, individual data points and to form the endpoints of other ranges. For example, a ratio in the range of about 1 to about 200 should be understood to include the explicitly recited limits of about 1 and about 200, but also to include individual ratios such as about 2, about 3, and about 4, sub-ranges such as about 1 to about 10, about 10 to about 50, about 20 to about 100, about 100 to about 200, and so forth, and related ranges such as greater than about 1 or less than about 200.

1. A sustainable biopolymer composition for skin-contact applications, comprising:
a polymer matrix comprising at least one gelling agent selected from the group consisting of polysaccharide-based polymers, plant-derived polymers, microbial polysaccharides, animal-derived polymers, and combinations thereof;
a humectant system comprising at least one sugar material selected from the group consisting of disaccharides, monosaccharides, sugar alcohols, oligosaccharides, and combinations thereof; and
water;
wherein the polymer matrix, the humectant system, and the water form a homogeneous gel network, and wherein the composition is configured to be shaped into a skin-contact article and dehydrated to form a biodegradable adhesive material.

2. The sustainable biopolymer composition of claim 1, wherein the gelling agent comprises at least one plant-derived polymer selected from the group consisting of agar, pectin, cellulose derivatives, such as, CMC (carboxymethyl cellulose), HPMC (hydroxypropyl methylcellulose), HPC, EC (ethyl cellulose), guar gum, locust bean gum, xanthan gum, konjac glucomannan, tara gum, psyllium husk polysaccharide, starch (native or modified), pullulan, inulin, and combinations thereof.

3. The sustainable biopolymer composition of claim 1, wherein the gelling agent comprises at least one animal-derived polymer selected from the group consisting of gelatin, collagen, chitosan, hyaluronic acid, fibrin, and combinations thereof.

4. The sustainable biopolymer composition of claim 1, wherein the gelling agent comprises at least one natural polysaccharide selected from the group consisting of carrageenan (kappa, iota, lambda), alginate (sodium alginate, calcium alginate), fucoidan, ulvan, and combinations thereof.

5. The sustainable biopolymer composition of claim 1, wherein the gelling agent comprises at least one microbial polysaccharide selected from the group consisting of gellan gum, dextran, levan, bacterial cellulose, and combinations thereof.

6. The sustainable biopolymer composition of claim 1, wherein the humectant system further comprises at least one sugar alcohol selected from the group consisting of sorbitol, mannitol, xylitol, erythritol, maltitol, isomalt, glycerol and propylene glycol and combinations thereof.

7. The sustainable biopolymer composition of claim 1, wherein the humectant system further comprises at least one saccharide selected from the group consisting of lactose, maltose, trehalose, isomaltulose, glucose, fructose, galactose, mannose, ribose, xylose, maltodextrin, fructooligosaccharides (FOS), galactooligosaccharides (GOS), and combinations thereof.

8. The sustainable biopolymer composition of claim 1, wherein the humectant system further comprises at least one biocompatible ionic liquid.

9. The sustainable biopolymer composition of claim 1, further comprising pyrrolidone carboxylic acid or a salt thereof.

10. The sustainable biopolymer composition of claim 1, further comprising a bioadhesive polymer component selected from the group consisting of chitosan derivatives, carboxyl-containing polymers, and combinations thereof.

11. The sustainable biopolymer composition of claim 1, further comprising at least one functional additive selected from the group consisting of fragrance compounds, menthol, nourishing oils, skin care actives, pimple treatment actives, moisturizing ingredients, and preservatives.

12. A multi-layer sustainable biopolymer article for skin-contact applications, comprising:
a structural backing layer, comprising a first composition comprising:
a gelling agent selected from the group consisting of polysaccharide-based polymers, plant-derived polymers, microbial polysaccharides, animal-derived polymers, and combinations thereof, and
a humectant material selected from the group consisting of disaccharides, monosaccharides, sugar alcohols, oligosaccharides, and combinations thereof,
wherein the structural backing layer is in a dehydrated state; and
a skin-contact adhesive layer, comprising a second composition comprising:
a gelling agent selected from the group consisting of polysaccharide-based polymers, plant-derived polymers, microbial polysaccharides, animal-derived polymers, and combinations thereof,
a humectant material selected from the group consisting of disaccharides, monosaccharides, sugar alcohols, oligosaccharides, and combinations thereof, and
wherein the gelling agent content of the skin-contact adhesive layer is greater than a gelling agent content of the structural backing layer, and
wherein the skin-contact adhesive layer is configured to contact skin and provide adhesion.

13. The multi-layer sustainable biopolymer article of claim 12, wherein at least the skin-contact adhesive layer comprises glycerin, and wherein where the structural backing layer comprises glycerin a glycerin content of the skin-contact adhesive layer is greater than a glycerin content of the structural backing layer.

14. The multi-layer sustainable biopolymer article of claim 12, further comprising a starch-based durability layer, the starch-based durability layer comprising tapioca starch.

15. The multi-layer sustainable biopolymer article of claim 14, further comprising a fabric layer positioned between the starch-based durability layer and the skin-contact adhesive layer, wherein the fabric layer comprises a transparent fabric.

16. The multi-layer sustainable biopolymer article of claim 12, further comprising a bioplastic layer, the bioplastic layer comprising starch and an organic acid.

17. A method of manufacturing a sustainable biopolymer material for skin-contact applications, comprising:
dissolving at least one gelling agent selected from the group consisting of polysaccharide-based polymers, plant-derived polymers, microbial polysaccharides, animal-derived polymers, and combinations thereof in water to form a solution;
adding at least one humectant material selected from the group consisting of disaccharides, monosaccharides, sugar alcohols, oligosaccharides, and combinations thereof to the solution;
heating the solution;
pouring the solution into a three-dimensional mold; and
dehydrating the molded solution by air drying to form the sustainable biopolymer material, wherein the dehydrating produces a first surface having greater tackiness and a second surface having greater structural stability.

18. The method of claim 17, further comprising cooling the solution and adding at least one plasticizer to the cooled solution while stirring prior to pouring the solution into the three-dimensional mold.

19. The method of claim 18, wherein the at least one gelling agent comprises at least one of agar and glycerin, and the method further comprises adding the humectant material while stirring, wherein the humectant agent at least comprises xanthan gum.

20. The method of claim 17, wherein the dehydrating comprises air drying for using the three-dimensional mold configured to expose one surface of the molded solution to air, wherein the exposed surface becomes the first surface having greater tackiness and a surface in contact with the mold becomes the second surface having greater structural stability.
